(19) United States
(12) Patent Application Publication
(10) Pub. No.: US 2026/0015299 A1
(43) Pub. Date: Jan. 15, 2026

(54) METHODS OF ALKYLATING ARENES

(71) Applicants: Saudi Arabian Oil Company, Dhahran (SA); King Abdullah University of Science and Technology, Thuwal (SA)

(72) Inventors: Robert Peter Hodgkins, Dhahran (SA); Omer Refa Koseoglu, Dhahran (SA); Rajesh Kumar Parsapur, Thuwal (SA); Anissa Bendjeriou-Sedjerari, Thuwal (SA); Kuo-Wei Huang, Thuwal (SA); Magnus Rueping, Thuwal (SA)

(73) Assignees: Saudi Arabian Oil Company, Dhahran (SA); King Abdullah University of Science and Technology, Thuwal (SA)

(21) Appl. No.: 18/772,711

(22) Filed: Jul. 15, 2024

(51) Int. Cl. C07C 2/66; B01J 29/06; B01J 35/50; B01J 35/63; B01J 35/64

(52) U.S. Cl. CPC C07C 2/66; B01J 29/06; B01J 35/50; B01J 35/633; B01J 35/635; B01J 35/638; B01J 35/647; C07C 2529/06

(57) ABSTRACT

A method for alkylating arenes may comprise: passing a feedstock comprising one or more arenes into a reactor; contacting the feedstock with a zeolitic material in the reactor, wherein: in the reactor the one or more arenes are alkylated to form one or more alkylated arenes; the zeolitic material has a *BEA framework and comprises mesopores; and at least a portion of the mesopores are arranged in cubic symmetry.

### FIELD

The embodiments described herein generally relate to chemical processing and, more particularly, to chemical processing using zeolites.

### BACKGROUND

Arene alkylation is an important industrial chemical process that produces a variety of useful and desired chemical compounds. For example, many alkylated arenes are used as precursors for a wide range of products such as, plastics, pharmaceuticals, and surfactants. The methods of alkylating arenes may utilize catalysts, such as zeolites to improve or enable the alkylation reaction.

### SUMMARY

It may be desirable to improve the methods of alkylating arenes. Embodiments of the present disclosure are directed to methods of alkylating arenes using zeolites. It has been discovered that using a zeolite beta that has long range mesoporous ordering with cubic symmetry may improve the conversion rate of an arene alkylation reaction when compared with a reaction that utilizes a zeolite beta that does not have such mesoporous ordering.

According to one or more embodiments, a method for alkylating arenes may comprise: passing a feedstock comprising one or more arenes into a reactor; contacting the feedstock with a zeolitic material in the reactor, wherein: in the reactor the one or more arenes are alkylated to form one or more alkylated arenes; the zeolitic material has a *BEA framework and comprises mesopores; and at least a portion of the mesopores are arranged in cubic symmetry.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawings and claims, or recognized by practicing the described embodiments. The drawings are included to provide a further understanding of the embodiments and, together with the detailed description, serve to explain the principles and operations of the claimed subject matter. However, the embodiments depicted in the drawings are illustrative and exemplary in nature, and not intended to limit the claimed subject matter.

### BRIEF DESCRIPTION OF THE DRAWINGS

The following detailed description of specific embodiments of the present disclosure can be best understood when read in conjunction with the following drawings in which:

FIG. 1A depicts the low angle X-Ray Diffraction (“XRD”) patterns of zeolite Beta particles with mesopores, according to one or more embodiments of the present disclosure;

FIG. 1B depicts the high angle XRD patterns of zeolite Beta particles with mesopores, according to one or more embodiments of the present disclosure;

FIG. 2A depicts the low angle XRD patterns of (a) calcined zeolite Beta particles with no mesopores and (b) calcined zeolite Beta particles with mesopores, according to one or more embodiments of the present disclosure;

FIG. 2B depicts the high angle XRD patterns of (a) calcined zeolite Beta particles with no mesopores and (b) calcined zeolite Beta particles with mesopores, according to one or more embodiments of the present disclosure;

FIG. 3 depicts N₂ physisorption isotherms of (a) zeolite Beta particles with no mesopores and (b) zeolite Beta particles with mesopores, according to one or more embodiments of the present disclosure; and

FIG. 4 depicts the conversion of an arene alkylation reaction performed with zeolite Beta particles with no mesopores and zeolite Beta particles with mesopores, according to one or more embodiments of the present disclosure.

Reference will now be made in greater detail to various embodiments, some embodiments of which are illustrated in the accompanying drawings.

### DETAILED DESCRIPTION

One or more embodiments presently described herein are directed to methods of alkylating arenes. The methods of the present disclosure may beneficially improve the conversion rate of the arene alkylation reaction when compared with conventional methods. As described herein, improved conversion may be a result of utilizing zeolitic materials as catalysts that include mesopores having cubic symmetry. Utilizing such zeolites may allow for the reactor operating conditions to be less severe (e.g., lower temperature and/or pressure and/or less catalyst and/or less residence time), improving chemical conversion efficiency.

In one or more embodiments, the method may include passing a feedstock comprising one or more arenes into a reactor. The arenes may comprise simple single aromatic ring arenes (e.g., benzene). The arenes may comprise single aromatic ring arenes having one or more hydrocarbon molecules bonded thereto (e.g., C₁-C₁₀ hydrocarbon groups), such as toluene, xylenes, or mesitylene. The arenes may comprise single aromatic ring arenes, with or without additional hydrocarbon groups (e.g., C₁-C₁₀ hydrocarbon groups) bonded thereto, and having additional functional groups bonded thereto (e.g., OH groups or halides), such as phenol, benzyl alcohol, chlorobenzene, or 2-methyl chlorobenze. The arenes may comprise alkyl or aryl-substituted mono/polyaromatics. The arenes may comprise heteroarenes, arenes having heteroatoms (e.g., oxygen, nitrogen, or sulfur) within the ring or functionalized thereto, such as pyridine, thiane, furan, or phenolates. Suitable arenes may also include benzofuran, naphthalene, naphthyl alcohol, and alkylated equivalents. It should be understood that the one or more arenes in the feedstock may include one or more of the aforementioned arenes. In one or more embodiments, the one or more arenes may comprise, consist essentially of, or consist of benzyl alcohol, mesitylene, or both. In one or more embodiments the feedstock may comprise from 0.1 wt. % to 100 wt. % of the one or more arenes, such as from 0.1 wt. % to 10 wt. %, from 10 wt. % to 20 wt. %, from 20 wt. % to 30 wt. %, from 30 wt. % to 40 wt. %, from 40 wt. % to 50 wt. %, from 50 wt. % to 60 wt. %, from 60 wt. % to 70 wt. %, from 70 wt. % to 80 wt. %, from 80 wt. % to 90 wt. %, from 90 wt. % to 100 wt. %, or any combination of two or more of these ranges.

In one or more embodiments, the feedstock may also comprise from 0.1 wt. % to 100 wt. % of an alkyl source, such as from 0.1 wt. % to 10 wt. %, from 10 wt. % to 20 wt. %, from 20 wt. % to 30 wt. %, from 30 wt. % to 40 wt. %, from 40 wt. % to 50 wt. %, from 50 wt. % to 60 wt. %, from 60 wt. % to 70 wt. %, from 70 wt. % to 80 wt. %, from 80 wt. % to 90 wt. %, from 90 wt. % to 100 wt. %, or any combination of two or more of these ranges. In one or more embodiments, the alkyl source may be a C₂ to C₂₀ hydrocarbon. In one or more embodiments, the alkyl source may comprise, consist essentially of, or consist of olefins, alcohols, thiols, or combinations thereof. In one or more embodiments, the alkyl source may comprise aliphactic alcohols, aromatic alcohols, or both aromatic and aliphatic alcohol. Generally, the choice of alkyl source may have a substantial impact on the activity and selectivity of the reaction.

In one or more embodiments the alkyl source and the one or more arenes may be passed into the reactor in a mixed stream. In other embodiments, the alkyl source and the one or more arenes may be passed into the reactor via separate streams and are mixed in the reactor. In embodiments where the alkyl source and the one or more arenes are passed into the reactor in a mixed stream, the mixed feedstock may be the product of a cracking unit, such as from a fluidized catalytic cracking unit, a thermal cracking unit, a steam cracking unit, or a delayed coking unit.

In one or more embodiments, the feedstock may have a molar ratio of arenes to alkyl source (e.g., alcohol) of from 2:1 to 25:1, such as from 2:1 to 5:1, from 5:1 to 8:1, from 8:1 to 11:1, from 11:1 to 14:1, from 14:1 to 17:1, from 17:1 to 20:1, from 20:1 to 22.5:1, from 22.5:1 to 25:1, or any combination of one or more of these ranges. Without being limited by theory, it is believed that reaction kinetics more strongly favor the alkylation pathway than the self-condensation pathway as the arene to alcohol ratio increases.

As described hereinabove, the methods of alkylating arenes of the present disclosure may also include contacting the feedstock with a catalyst comprising zeolitic material in the reactor. In the reactor the one or more arenes may be alkylated to form one or more alkylated arenes. In one or more embodiments, the reactor may be a fixed-bed reactor, a moving-bed reactor, an ebullated-bed reactor, or a continuous stirred-tank reactor.

In one or embodiments the temperature of the reactor is from 15° C. to 300° C., such as from 15° C. to 30° C., from 30° C. to 45° C., from 45° C. to 60° C., from 60° C. to 75° C., from 75° C. to 90° C., from 90° C. to 105° C., from 105° C. to 120° C., from 120° C. to 135° C., from 135° C. to 150° C., from 150° C. to 165° C., from 165° C. to 180° C., from 180° C. to 195° C., from 195° C. to 210° C., from 210° C. to 225° C., from 225° C. to 240° C., from 240° C. to 255° C., from 255° C. to 270° C., from 270° C. to 285° C., from 285° C. to 300° C., or any combination of one or more of these ranges.

In one or more embodiments, the pressure in the reactor may be from 1 kg/cm² to 120 kg/cm², such as from 1 kg/cm² to 10 kg/cm², from 10 kg/cm² to 20 kg/cm², from 20 kg/cm² to 30 kg/cm², from 30 kg/cm² to 40 kg/cm², from 40 kg/cm² to 50 kg/cm², from 50 kg/cm² to 60 kg/cm², from 60 kg/cm² to 70 kg/cm², from 70 kg/cm² to 80 kg/cm², from 80 kg/cm² to 90 kg/cm², from 90 kg/cm² to 100 kg/cm², from 100 kg/cm² to 110 kg/cm², from 110 kg/cm² to 120 kg/cm², or any combination of one or more of these ranges.

In one or more embodiments, the residence time is from 15 minutes to 240 minutes, such as from 15 minutes to 30 minutes, from 30 minutes to 45 minutes, from 45 minutes to 60 minutes, from 60 minutes to 75 minutes, from 75 minutes to 90 minutes, from 90 minutes to 105 minutes, from 105 minutes to 120 minutes, from 120 minutes to 135 minutes, from 135 minutes to 150 minutes, from 150 minutes to 165 minutes, from 165 minutes to 180 minutes, from 180 minutes to 195 minutes, from 195 minutes to 210 minutes, from 210 minutes to 225 minutes, from 225 minutes to 240 minutes, or any combinations of one or more of these ranges. As used herein, residence time refers to the contact time of the catalyst with the arenes at the reaction temperature.

In one or more embodiments, the liquid hourly space velocity (LHSV) in the reactor during arene alkylation may be from 0.1 h⁻¹ to 6 h⁻¹, such as from 0.1 h⁻¹ to 0.5 h⁻¹, from 0.5 h⁻¹ to 1 h⁻¹, from 1 h⁻¹ to 1.5 h⁻¹, from 1.5 h⁻¹ to 2 h⁻¹, from 2 h 1 to 2.5 h⁻¹, from 2.5 h⁻¹ to 3 h⁻¹, from 3 h⁻¹ to 3.5 h⁻¹, from 3.5 h⁻¹ to 4 h⁻¹, from 4 h⁻¹ to 4.5 h⁻¹, from 4.5 h⁻¹ to 5 h⁻¹, from 5 h⁻¹ to 5.5 h⁻¹, from 5.5 h⁻¹ to 6 h⁻¹, or any combination of one or more of these ranges.

In one or more embodiments, the arene concentration in the reactor may be from 0.1 wt. % to 70 wt. %, on the basis of all organic compounds in the reactor. For example, the arene concentration in the reactor may be from 0.1 to 1 wt. %, from 1 wt. % to 5 wt. %, from 5 wt. % to 10 wt. %, from 10 wt. % to 20 wt. %, from 20 wt. % to 40 wt. %, from 40 wt. % to 60 wt. %, from 60 wt. % to 80 wt. %, from 80 wt. % to 100 wt. %, or any combination of two or more of these ranges.

As described hereinabove, the one or more arenes may be alkylated in the reactor to form one or more alkylated arenes, such as alkyl or aryl-substituted polyaromatics. In one or more embodiments the one or more alkylated arenes may comprise one or more of 1,3,5-trimethyl-2-benzylbenzene and dibenzyl ether.

In one or more embodiments the alkylated arenes may be passed out the reactor as a product stream. The product stream may comprise from 0.1 wt. % to 100 wt. % of the one or more alkylated arenes, such as from 0.1 wt. % to 80 wt. %, from 0.1 wt. % to 10 wt. %, from 10 wt. % to 20 wt. %, from 20 wt. % to 30 wt. %, from 30 wt. % to 40 wt. %, from 40 wt. % to 50 wt. %, from 50 wt. % to 60 wt. %, from 60 wt. % to 70 wt. %, from 70 wt. % to 80 wt. %, from 80 wt. % to 90 wt. %, from 90 wt. % to 100 wt. %, or any combination of one or more of these ranges.

According to one or more embodiments, in the reactor, the one or more arenes are contacted with a catalyst comprising a zeolitic material. In embodiments, the catalyst comprises at least 80 wt. %, at least 90 wt. %, at least 95 wt. %, at least 99 wt. %, at least 99.9 wt. %, or even at least 99.99 wt. % of the zeolitic material. As used throughout this disclosure, “zeolitic material” or “zeolites” generally refer to micropore-containing inorganic materials with regular intra-crystalline cavities and channels of molecular dimension, as would be understood by those skilled in the art. Zeolites generally comprise a crystalline structure, as opposed to an amorphous structure. The microporous structure of zeolites may render large surface areas and desirable size-/shape-selectivity, which may be advantageous for catalysis.

Generally, zeolites may be characterized by a microporous framework type, which defines their microporous structure. Framework types are described in, for instance, “Atlas of Zeolite Framework Types” by Christian Baerlocher et al., Sixth Revised Edition, published by Elsevier, 2007, the teachings of which are incorporated by reference herein. As used in this disclosure, “zeolite Beta” refers to zeolite having a *BEA framework type according to the IZA zeolite nomenclature and consisting majorly of silica and/or alumina, as would be understood by one skilled in the art. The *BEA microporous framework has a three-dimensional network of 12-membered ring pores featuring an intergrowth of two or more polymorphs with pore diameters of 0.56 nm×0.56 nm and 0.66 nm×0.67 nm. In embodiments, the micropores of the zeolite Beta particles disclosed herein may have diameters of greater than or equal to 0.1 nm and less than or equal to 2 nm.

In one or more embodiments, the catalysts, the zeolitic materials, or both described herein may be shaped as particles. In embodiments, the particles may be generally spherical or irregularly globular (that is, non-spherical). In embodiments, the particles disclosed herein have a “particle size” that may be measured as the greatest distance between two points located on a single zeolite particle. For instance, the particle size of a spherical particle would be its diameter. In other shapes, the particle size may be measured as the distance between the two most distant points of the same particle, wherein these points may lie on outer surfaces of the particle.

Particle size may be determined by, for instance, visual examination under a microscope, or by dynamic light scattering (“DLS”) whereby the hydrodynamic radius is obtained. The particle size may be an average particle size. In embodiments, the particles disclosed herein may have an average particle size from 20 nm to 5000 nm, from 20 nm to 50 nm, from 50 nm to 100 nm, from 100 nm to 200 nm, from 200 nm to 400 nm, from 400 nm to 600 nm, from 600 nm to 800 nm, from 800 nm to 1000 nm, from 1000 nm to 2000 nm, from 2000 nm to 3000 nm, from 3000 nm to 4000 nm, from 4000 nm to 5000 nm, or any combination of one or more of these ranges.

In one or more embodiments, the zeolitic material may have an average micropore volume and/or an average mesopore volume, where the total pore volume is the sum of these two. The mesopore and micropore volumes may be calculated according to the Barrett-Joiner-Halenda (BJH) method of determining mesopore volume known to one having skill in the art. Details regarding the t-plot method and the BJH method of calculating micropore volume and mesopore volume respectively are provided in Galarneau et al., “Validity of the t-plot Method to Assess Microporosity in Hierarchical Micro/Mesoporous Materials”, Langmuir 2014, 30, 13266-13274, for example. In one or more embodiments, the zeolitic material may have a total pore volume of from 0.01 ml/g to 1.5 ml/g, such as from 0.01 ml/g to 0.1 ml/g, from 0.1 ml/g to 0.2 ml/g, from 0.2 ml/g to 0.3 ml/g, from 0.3 ml/g to 0.4 ml/g, from 0.4 ml/g to 0.5 ml/g, from 0.5 ml/g to 0.6 ml/g, from 0.6 ml/g to 0.7 ml/g, from 0.7 ml/g to 0.8 ml/g, from 0.8 ml/g to 0.9 ml/g, from 0.9 ml/g to 1 ml/g, from 1 ml/g to 1.1 ml/g, from 1.1 ml/g to 1.2 ml/g, from 1.2 ml/g to 1.3 ml/g, from 1.3 ml/g to 1.4 ml/g, from 1.4 ml/g to 1.5 ml/g, or any combination of one or more of these ranges.

In one or more embodiments, the *BEA microporous framework of the zeolitic material described herein may comprise silica and/or alumina, which is consistent with the materials included in a zeolite Beta, as is understood by those skilled in the art. The ratio of silica to alumina in zeolite Beta may vary. According to one or more embodiments described herein, the molar ratio of silica to alumina in the zeolite Beta may be from 15 to 1500, such as from 15 to 100, from 100 to 200, from 200 to 300, from 300 to 400, from 400 to 500, from 500 to 600, from 600 to 700, from 700 to 800, from 800 to 900, from 900 to 1000, from 1000 to 1100, from 1100 to 1200, from 1200 to 1300, from 1300 to 1400, from 1400 to 1500, or any combination of one or more of these ranges. In some embodiments, the *BEA microporous framework may be a siliceous only framework and may essentially consist of silica. In some embodiments, the *BEA microporous framework may comprise at least 80 wt. %, at least 90 wt. %, at least 95 wt. %, at least 99 wt. %, or even at least 99.9 wt. % of the combined weight of silica and alumina, on the basis of the total weight of the zeolitic material.

In one or more embodiments, the zeolitic materials disclosed herein may comprise a plurality of mesopores. In some embodiments, the plurality of mesopores may have diameters greater than or equal to 2 nm, such as greater than or equal to 2 nm and less than or equal to 50 nm, such as from 2 nm to 5 nm, from 5 nm to 10 nm, from 10 nm to 15 nm, from 15 nm to 20 nm, from 20 nm to 25 nm, from 25 nm to 30 nm, from 30 nm to 35 nm, from 35 nm to 40 nm, from 40 nm to 45 nm, from 45 nm to 50 nm, or any combination of one or more of these ranges. In embodiments, the zeolitic materials may comprise a plurality of micropores, each having a diameter of less than 2 nm.

In embodiments, zeolitic materials may have a surface area of about 200-1500 m²/g, 200-1000 m²/g, 200-900 m²/g, 400-1500 m²/g, 400-1000 m²/g, 400-900 m²/g, 500-1500 m²/g, 500-1000 m²/g, or 500-900 m²/g.

In one or more embodiments, the zeolitic materials may be hierarchically ordered, such that they include both micropores and mesopores. Hierarchically ordered zeolitic materials may have a microporous bulk phase with a plurality of mesopores disposed therein. The microporous bulk phase extending between the mesopores may be referred to as a “mesophase”. The channels of the mesopores may comprise the micropores bulk phase and may be microporous.

In one or more embodiments, at least a portion of the mesophase comprising the mesopores may be arranged in cubic symmetry (also referred to herein as a “cubic mesophase”). As referred to herein, “cubic symmetry” refers to a unit structure of a cube. Generally, when a material comprises mesopores arranged in cubic symmetry, the mesopores are arranged and spaced such that the centers of adjacent mesopores will form the exterior corners of a cube when viewed from at least one direction. In embodiments, the zeolitic material as a whole, the cubic mesophase, or both has an Ia-3d mesopore structure. The presence of mesopores arranged in cubic symmetry can be determined by those skilled in the art. For example, the presence of mesopores arranged in cubic symmetry may be determined by secondary peaks associated with the periodic arrangement of mesopores in x-ray diffraction (XRD) reflections for the given mesosphere (as described in U.S. Pat. Appln. No. U.S. 2024/0009656 A1), and/or by observations in microscopy. (Examples of high resolution transmission electron microscopy (HR-TEM) micrographs of zeolitic materials having cubic symmetry as disclosed in FIGS. 5A and 5B of US2024/0010935A1).

In embodiments, the zeolitic materials may have mesoporous ordering of 3D-cubic symmetry. A material having mesoporous ordering of 3D-cubic symmetry refers to a material where the mesopores are arranged and spaced such that the centers of four adjacent mesopores will form the exterior corners of a cube when viewed from at least three directions (e.g., X, Y, and Z directions). The presence of mesoporous ordering of 3D-cubic symmetry can be determined by those skilled in the art. For example, the presence of mesoporous ordering of 3D-cubic symmetry may be determined by secondary peaks associated with the periodic arrangement of mesopores in x-ray diffraction (XRD) reflections for the given mesosphere (as described in U.S. Pat. Appln. No. U.S. 2024/0009656 A1), and/or by observations in microscopy. (Examples of TEM micrographs of zeolitic materials having cubic symmetry as disclosed in FIGS. 5A and 5B of US2024/0010935A1).

The zeolitic materials may have long-range mesoporous ordering of cubic symmetry. For example, the zeolitic materials may have mesopores arranged in a cubic unit structure repeating over a length of greater than about 50 nm, greater than 75 nm, or even greater than 100 nm. For examples, the zeolitic materials may have mesopores arranged in a cubic unit structure repeating over at least 10, such as at least 50, at least 100, at least 500, or at least 1000 connected cubic unit cells. The mesopores may repeat over a length of greater than about 10 nm, such as 50 nm, greater than 75 nm, or even greater than 100 nm; over at least 10, such as at least 50, at least 100, at least 500, or at least 1000 connected cubic unit cells; or both.

Long-range mesoporous ordering may be defined by secondary peaks associated with the periodic arrangement of mesopores in x-ray diffraction (XRD) patterns for the given mesosphere, and/or by observations in microscopy. These peaks associated with the mesoporous traits in the zeolitic materials may be observed at low 2-theta angles, such as less than 7.5°, such as from 0.5° to 7.5°, from 0.5° to 1°, from 1° to 2°, from 2° to 3°, from 3° to 4°, from 4° to 5°, from 5° to 6°, from 6° C. to 7.5° C., or any combination of two or more of these ranges. The zeolitic material may also exhibits high-angle peaks associated with the zeolitic materials and are observed at high 2-theta angles. In embodiments, the zeolitic material is characterized by mesopores arranged in cubic symmetry. In embodiments, the zeolitic material is a 3D-cubic ordered mesoporous zeolite. Zeolitic materials where the mesopores arranged in cubic symmetry may be characterized by cubic mesoporous channels with micropore channels in the walls of the mesostructure and the mesopores. The mesopores may be arranged in one of Ia-3d, Fm-3m, Pm-3n, Pn-3m, or Im-3m symmetry. In embodiments, the mesopores are arranged in Ia-3d symmetry and secondary XRD peaks associated with the periodic arrangement of mesopores are present at one or more of the (220), (321), (400), (420) and (332) reflections. In some embodiments, such as those where the material has Ia-3d symmetry, the zeolitic material may show the presence of three-dimensional extension of bicontinuous gyroidal mesoporous channels. In embodiments, the mesopores are arranged in Ia-3d symmetry and the long-range mesoporous arrangement in Ia-3d symmetry is observable by microscopy viewed by the electron beam down a suitable zone axis, for example the [311], [111]m or zone axes. In embodiments, the mesopores are arranged in Fm-3m symmetry and Fm-3m symmetry is observable by microscopy viewing an electron beam down a suitable axis, such as a or a zone axis.

The zeolitic materials may be arranged in a cubic symmetry on the meso-scale regardless of their atomic-level symmetry or structure. Accordingly, zeolitic materials having a cubic mesophase includes zeolitic materials characterized by atomic-level symmetry and possessing micropores that are inherent to *BEA framework materials, arranged in a cubic symmetry at the meso-scale level with mesopores, wherein walls of the mesopores and a mass of the mesostructure between mesopores is characterized by said zeolite. In one embodiment a zeolitic material includes a *BEA framework zeolite having atomic-level tetragonal symmetry arranged in a cubic symmetry meso-scale.

Suitable zeolitic materials having a *BEA framework include those prepared according to U.S. Pat. Appln. Pub. No. 2024/0009656 A1, the entirety of which is incorporated herein by reference.

The catalyst may have a Lewis acidity and a Brønsted acidity. The catalyst may have a Lewis acidity in the range of from 25 to 400 μmol/g, such as from 75 to 250 μmol/g, from 25 to 50 μmol/g, from 50 to 75 μmol/g, from 75 to 100 μmol/g, from 100 to 150 μmol/g, from 150 to 200 μmol/g, from 200 to 250 μmol/g, from 250 to 300 μmol/g, from 300 to 350 μmol/g, from 350 to 400 μmol/g, or any combination of two or more of these ranges. Generally, the amount of Lewis acidity correlates with the acid strength and acid site-density, which in turn, help to control the activity and selectivity values. The catalyst may have a Bronsted acidity in the range of from 10 to 150 μmol/g, such as from 10 to 100 μmol/g, from 10 to 25 μmol/g, from 25 to 50 μmol/g, from 50 to 75 μmol/g, from 75 to 100 μmol/g, from 100 to 125 μmol/g, from 125 to 150 μmol/g, or any combination of two or more of these ranges. Generally, the amount of Brønsted acidity correlates with the acid strength and acid site-density, which in turn, help to control the activity and selectivity values. The Lewis and Brønsted acidity can be determined by Fourier Transform Infra-Red (FTIR) spectroscopy using pyridine adsorption techniques or by ammonia-temperature programmed desorption (TPD)

The catalyst may have a ratio of Lewis acidity to Brønsted acidity of from 0.25 to 40, such as from 0.75 to 25, from 0.25 to 5, from 0.5 to 1, from 1 to 2.5, from 2.5 to 5, from 5 to 10, from 10 to 15, from 15 to 20, from 20 to 25, from 25 to 30, from 30 to 35, from 35 to 40, or any combination of two or more of these ranges. Generally, the ratio of Lewis acidity to Brønsted acidity correlates with the acid strength and acid site-density, which in turn, help to control the activity and selectivity values.

### EXAMPLES

The various embodiments of methods described will be further clarified by the following examples. The examples are illustrative in nature, and should not be to limit the subject matter of the present disclosure.

Micropore surface area (S_{mic}), total surface area (Sₜₒₜ), and total pore volume (Vₜₒₜ) were determined with the Brunauer-Emmett-Teller (BET) method in the P/P₀ range from 0.1 to 0.3. The t-plot method was used to estimate the micropore volume (V_{mic}). Nitrogen physisorption measurements were performed at −196° C. on a Micromeritics ASAP 2420 porosimeter. The mesopore size distribution (D) was obtained using a DFT model applied to the adsorption branch of the N₂ physisorption isotherm.

X-ray diffraction (XRD) measurements were taken using a Bruker D8-Twin diffractometer having Cu Kα radiation (λ=0.154 nm) detector. Data was recorded in the range of from 0.5 to 50 2θ/degrees. The voltage and current were 40 kV and 40 mA, respectively, and the scanning rate was 0.5 degree min⁻¹.

### Example 1—Zeolite Synthesis

Initially, 1.2 g of urea was dissolved in 60.0 g of water to form a homogeneous solution. To this mixture, 2.0 g of dried zeolite, CP-811T-100, available from Zeolyst International, (SiO₂/Al₂O₃ ratio of about 100) was added and stirred for 15 min. Subsequently, 0.2 g of NH₄NO₃, and 3.0 mL of dimethyloctadecyl(3-trimethoxysilylpropyl) ammonium chloride (DOAC, 42.0 wt % in methanol) were added stepwise and the mixture was further stirred for 2 hours at room temperature. The resultant solution was hydrothermally treated at 150° C. for 36 hours. The obtained solids were filtered, washed with water, and dried at 120° C. for 24 h. The synthesized products were calcined in air at 550° C. for 6 h at a ramp rate of 60° C. h⁻¹ to yield Sample 1 (S-1).

S-1 and Comparative Sample A (CS-A), which was CP-811T-100 used as received were examined and the physical characteristics of the zeolites was recorded in Table 1.

TABLE 1

| | S-1 | CS-A |
|---|---|---|
| S_{mic} (m²g⁻¹) | 345 | 459 |
| Sₜₒₜ (m²g⁻¹) | 715 | 623 |
| D (nm) | 4.2 | — |
| V_{mic} (cm³g⁻¹) | 0.14 | 0.18 |
| Vₜₒₜ (cm³g⁻¹) | 0.49 | 0.25 |

The data in Table 1 indicates that the example zeolite Beta particles disclosed herein has a higher total surface area (715 m²/g for S-1) than the conventional zeolite Beta particles (623 m²/g for CS-A). The example zeolite Beta particles also have a higher total pore volume (0.49 cm³/g for S-1) than the conventional zeolite Beta particles (0.25 cm³/g for CS-A). Because the microporous surface area and the microporous volume of S-1 are both approximate to or lower than the microporous surface area and the microporous volume of CS-A, it follows that the increase in total surface area and total pore volume is due to the presence of the mesopores in the example zeolite Beta particles. The Si/Al ratio of S-1 was 46.4 and the Si/Al ratio of CS-A was 46.

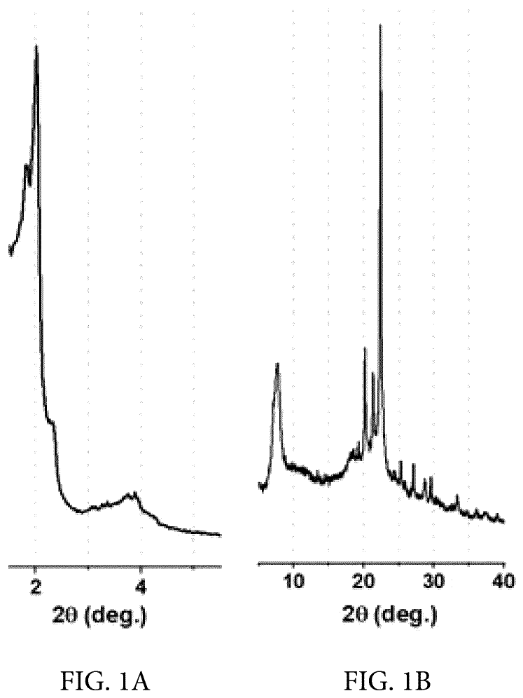

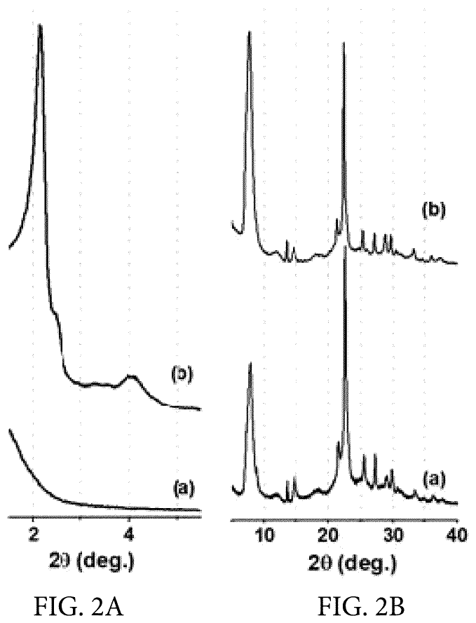

FIGS. 1A and 1B are the low angle (1A) and high angle (1B) XRD patterns for the as-made example zeolite Beta particles, S-1. FIGS. 2A and 2B are the low angle (2A) and high angle (2B) XRD patterns for the calcined example zeolite Beta particles (which include both the *BEA framework and the mesopores formed into the *BEA framework), S-1 (b), and the commercially available CS-A (a). In contrast to the CS-A, the XRD pattern of example zeolite S-1 shows reflections consistent with Ia3d cubic symmetry in the low-angle region, which suggests that the mesopores are organized uniformly amidst the crystal domain. The high angle XRD patterns of the mesoporous zeolite beta is analogous to the parent zeolite beta without any amorphous phases and impurities suggesting that the zeolite structure is retained during the post-synthetic modification process. The presence of low-angle XRD patterns after calcination indicate that the mesopore structure is thermally stable and the mesostructure is retained after the removal of surfactant.

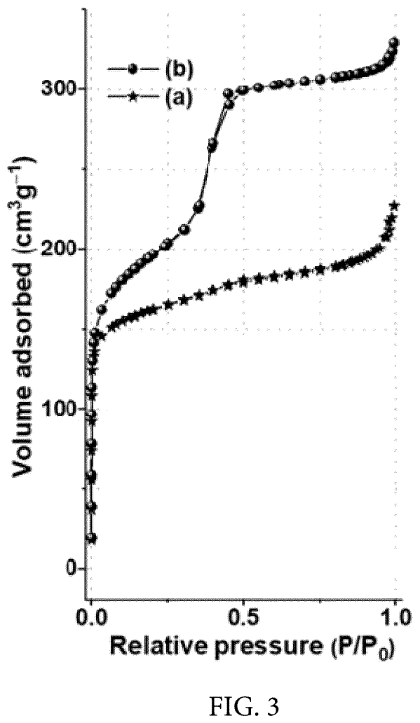

FIG. 3 N₂ physisorption isotherms for S-1 (b) and CS-A (a). Evaluation of the adsorption and desorption branches of these isotherms and the hysteresis between them reveals that the mesoporous zeolite Beta possess a type-IV isotherm with narrow hysteresis ranging over from 0.45 to 0.55 P/P₀, indicating the presence of uniform mesopores. In addition, the high N₂ adsorption below 0.1 P/P₀ suggests that the prepared structure also possesses a high amount of microporosity. In contrast, CS-A generates a type-I isotherm, which indicates a microporous structure without mesopores.

### Example 2—Arene Alkylation

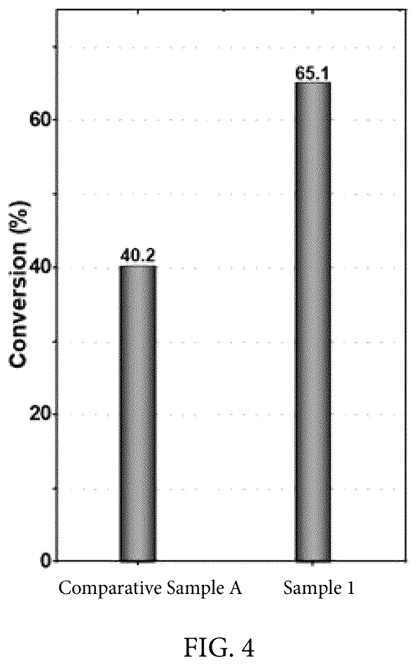

In example 2 the two zeolites from Example 1 were tested to determine their arene alkylation performance. In Example 2, 25 mg of the tested zeolite was contacted with a feed comprising benzyl alcohol/mesitylene in a molar ratio of 2.88/32.34 (0.089) Generally, mesitylene is generally used in excess amounts to avoid self-condensation of benzyl alcohol. The liquid phase alkylation of mesitylene was carried out in a two-necked round bottom (RB) flask connected to a reflux condenser and septum stopper. Initially, 25 mg of catalyst, 0.4 g dodecane (internal standard), and 32.34 mmol of mesitylene were added to the RB and stirred at 110° C. for 15 min. Later, 2.88 mmol of benzyl alcohol is added through the septum stopper to start the reaction. After 4 h, the reaction mixture was cooled under air and filtered with a PTFE syringe filter (0.45 microns). Thus, obtained reaction mixture was analyzed by a gas chromatograph (Agilent 7890A) with an HP-5 column. The results of the reactions are shown in FIG. 4.

As shown in FIG. 4, S-1 had significantly improved conversion when compared with CS-A. In particular, there is a 62% increase in the conversion rate when using the zeolite of S-1 when compared with the zeolite of CS-A.

What is claimed is:

1. A method for alkylating arenes the method comprising:
passing a feedstock comprising one or more arenes into a reactor; and
contacting the feedstock with a zeolitic material in the reactor, wherein:
in the reactor the one or more arenes are alkylated to form one or more alkylated arenes;
the zeolitic material has a *BEA framework and comprises mesopores; and
at least a portion of the mesopores are arranged in cubic symmetry.

2. The method of claim 1, wherein the feedstock comprises from 50 wt. % to 95 wt. % of the one or more arenes.

3. The method of claim 1, wherein the one or more arenes comprises one or more single aromatic ring arenes.

4. The method of claim 1, wherein the one or more arenes comprises of one or more of benzyl alcohol and mesitylene.

5. The method of claim 1, wherein the feedstock further comprises one or more olefins.

6. The method of claim 1, wherein an arene to alcohol weight ratio in the feedstock is from 0.5 to 25.

7. The method of claim 1, wherein the reactor is at a temperature of from 15° C. to 300° C.

8. The method of claim 1, wherein the feedstock is produced by a cracking unit.

9. The method of claim 1, wherein the liquid hourly space velocity in the reactor is from 0.1 h⁻¹ to 6 h⁻¹.

10. The method of claim 1, wherein the pressure within the reactor is from 1 kg/cm² to 120 kg/cm².

11. The method of claim 1, wherein a weight ratio of zeolitic material/benzyl alcohol in the reactor is from 0.025 to 1.

12. The method of claim 1, wherein the portion of the mesopores that are arranged in cubic symmetry are arranged in Ia-3d, Fm-3m, Pm-3n, Pn-3m or Im-3m symmetry.

13. The method of claim 12, wherein the portion of the mesopores that are arranged in cubic symmetry are arranged in Ia-3d symmetry and secondary peaks in XRD are present at one or more of (220), (321), (400), (420), or (332) reflections.

14. The method of claim 12, wherein the portion of the mesopores that are arranged in cubic symmetry are arranged in Ia-3d symmetry and the symmetry is observable by microscopy viewing an electron beam down a [311], [111], or zone axis.

15. The method of claim 12, wherein the portion of the mesopores that are arranged in cubic symmetry are arranged in Fm-3m symmetry and symmetry is observable by microscopy viewing an electron beam down a or a zone axis.

16. The method of claim 1, wherein the zeolitic material has a peak at two theta angles of less than or equal to 7.5° as measured using x-ray diffraction.

17. The method of claim 1, wherein the zeolitic material has a peak at two theta angles of from 0.5° to 7.5° as measured using x-ray diffraction.

18. The method of claim 1, wherein the zeolitic material has a total pore volume of from 0.01 to 1.5 ml/g.

19. The method of claim 1, wherein the zeolitic material comprises mesopores having a pore size of from 2 nm to 50 nm.

20. The method of claim 1, wherein the zeolitic material has a silica to alumina molar ratio of from 15 to 1500.
